(19) United States
(12) Patent Application Publication
(10) Pub. No.: US 2026/0028579 A1
(43) Pub. Date: Jan. 29, 2026

(54) DEVICE, SYSTEM AND PROCESS FOR THE ENHANCED PRODUCTION OF MANNOSYLERYTHRITOL LIPIDS (MELS) INTEGRATING FERMENTATION AND PRODUCT SEPARATION FROM FERMENTATION BROTH BY NON- INVASIVE METHODS

(71) Applicant: INSTITUTO SUPERIOR TÉCNICO, Lisboa (PT)

(72) Inventors: Frederico CASTELO ALVES FERREIRA, Lisboa (PT); Petar KEKOVIC, Lisboa (PT); Nuno Ricardo TORRES FARIA, Lisboa (PT); Miguel FIGUEIREDO NASCIMENTO, Lisboa (PT)

(21) Appl. No.: 18/997,138

(22) PCT Filed: Jul. 18, 2023
(86) PCT No.: PCT/PT2023/050020

§ 371 Date: Jan. 20, 2025

(30) Foreign Application Priority Data
Jul. 19, 2022 (PT) 118115

(51) Int. Cl. C12M 1/00; B01D 61/02; B01D 61/14; B01D 61/58; C12P 7/6481; C12P 19/44

(52) U.S. Cl. CPC C12M 47/10; B01D 61/58; C12P 7/6481; C12P 19/44; B01D 61/027; B01D 61/145; B01D 61/147

(57) ABSTRACT

Device (1) and system for macrofiltration are disclosed. The macrofiltration comprises mannosylerythritol lipids (MELs) recovery, the MELs being in the form of particles. System comprising the device (1) is also disclosed. The system comprises a first pathway (6) in fluid communication, wherein this first pathway comprises sequentially a first valve (3), a first pump (4), a bioreactor (2) and a second valve (10), and where this first pathway is between the at least one outlet and inlet of the device (1). Process for enhancing mannosylerythritol lipids (MELs) production is also disclosed.

### FIELD OF THE INVENTION

The present disclosure, in some embodiments thereof, relates to mannosylerythritol lipids (MELs), processes and devices for their production and separation.

### BACKGROUND OF THE INVENTION

Mannosylerythritol lipids (MELs) are a group of extracellular glycolipids with tensioactive properties. Those have a range of potential applications in various areas (medicine, cosmetics, pharmaceuticals, personal hygiene, bioremediation and cleaning, etc.) and their production and yeast strains that produce them are already established.

MELs consists of a molecule consisting of a hydrophilic part, composed of mannose and erythritol, and a hydrophobic part, composed of fatty acids carbon chains with a typical size that can go from 4 to 18 carbon atoms. The MELs can be acetylated, doubly acetylated or without acetylation in the mannose moiety. These different configurations of the hydrophilic and hydrophobic part contain different structures and properties.

There are several yeasts known to produce MELs, however, the strains Moesziomyces antarcticus and M. bullatus are those for which higher MELs productions are reported, with a maximum reported production of 165 g_{crude}/L of a product obtained in fermentations of M. bullatus, made of a crude comprising MELs and large amounts of unconsumed soybean oil (SBO) used as substrate together with glucose, yeast extract, and other components of the medium.

Fermentations for MELs production begin with a phase of cell growth after which the MELs production rate increases significantly, mainly in the presence of hydrophobic substrate (added in batch fermentation or batch feeding strategies). It is reported that MELs production can last up to 240-336 hours. The more efficient method reported in the literature used for the separation of MELs from the fermentation broth includes successive cumbersome multiple liquid-liquid extraction using several organic solvents (ethyl acetate, hexane, ethanol).

Typical MELs productions of high titers uses large amounts of vegetable oil (80-240 g/L), namely soybean oil, which are not fully consumed and thus are left as product contaminants. Separation of MELs from such triacylglycerols and other lipidic impurities by a solvent extraction relying in a single solvent has not yet been reported in literature, as MELs purification usually requires the combination of several solvents in multiple solvent steps.

Patent JP2008152690A discloses a method for producing mannosylerythritol lipids by culturing a microorganism capable of producing mannosylerythritol lipids in a culture solution containing a nutrient and a carbon source, the microorganism capable of producing mannosylerythritol lipids is Pseudozyma tsukubaensis.

Patent US20220127563A1 discloses an improved method for producing mannosylerythritol lipids (MELs) using yeasts not previously known to produce MELs. In particular, Meyerozyma guilliermondii (Pichia guilliermondii) is cultivated in a specially-tailored nutrient medium and under cultivation conditions such that the yeast unnaturally produces MELs and/or MELs-like molecules in greater amounts and at increased rates than when using standard MELs production with, for example, Pseudozyma aphidis.

There is still a need for methods for the production and recovery of MELs with higher efficiency, lower fermentation time, and reduced use of solvents.

### SUMMARY OF THE INVENTION

In one aspect of the present disclosure, there is provided a device (1) for macrofiltration comprising a grid (20), the grid comprising a plurality of channels, each channel having a width between 0.3 mm and 10 mm; the grid separating a first chamber and a second chamber, each chamber positioned on an opposite side of the grid (20); at least one inlet (22) and at least one outlet (24), wherein: the at least one inlet (22) is configured to evenly supply a fluid to the first chamber; the second chamber is configured to collect a filtrated fluid; and the at least one outlet (24) is configured to connect the second chamber to a bioreactor (2).

In a further embodiment, the macrofiltration comprises mannosylerythritol lipids (MELs) recovery.

In a further embodiment, the MELs are in the form of particles, the particles being retained in the first chamber.

In another aspect of the disclosure, there is provided a system comprising the device of the present disclosure.

In a further embodiment, the system further comprises a bioreactor (2).

In a further embodiment, the system comprises a first pathway (6) in fluid communication, wherein the first pathway comprises sequentially a first valve (3), a first pump (4), the bioreactor (2) and a second valve (10), and the first pathway is between the at least one outlet and inlet of the device (1).

In a further embodiment, the system further comprises a reservoir (5).

In a further embodiment, the system comprises a second pathway (7) in fluid communication, wherein this second pathway comprises sequentially a third valve (12), a second pump (8), the reservoir (5) and a fourth valve (14), and this second pathway is positioned between the at least one outlet and inlet of the device (1).

In a further embodiment, the system comprises: a bioreactor (2), a reservoir (5), a first pathway (6) and a second pathway (7), wherein the device (1) comprises at least one outlet (24) in fluid communication with the first pathway (6) and the second pathway (7), and at least one inlet (22) in fluid communication with the first pathway (6) and the second pathway (7); the first pathway (6) comprises sequentially a first valve (3), a first pump (4), the bioreactor (2) and a second valve (10), positioned between the at least one outlet and inlet of the device (1); and the second pathway (7) comprises sequentially a third valve (12), a second pump (8), the reservoir (5) and a fourth valve (14), positioned between the at least one outlet and inlet of the device (1).

In another aspect of the disclosure, there is provided a process for enhancing mannosylerythritol lipids (MELs) production, comprising the steps of: a) contacting between 1% and 70% weight per weight (w/w) of a substrate and between 0.01% and 10% of a yeast in a culture medium solution under fermentation conditions thereby producing MELs a in fermentation broth; b) separating the MELs from the fermentation broth, and c) washing the MELs with a second solution, wherein the ratio of the second solution to the fermentation broth is between 3:10 to 1:50 per volume (v/v), thereby enhancing mannosylerythritol lipids (MELs) production.

In a further embodiment, the second solution comprises an alcoholic solution and/or an aqueous solution.

In a further embodiment, the process is performed in the device of the present disclosure or in the system of the present disclosure.

In a further embodiment, the process further comprises a step of circulating the fermentation broth from step b), back to step a).

In a further embodiment, the MELs has a purity of more than 55%.

In a further embodiment, the MELs are in the form of particles.

In a further embodiment, the particles have a particle size of more than 1 mm.

In a further embodiment, the particles have a particle size between 1 mm and 50 mm.

In a further embodiment, the substrate comprises a hydrophilic carbon source, hydrophobic carbon source, or both.

In a further embodiment, the hydrophilic carbon source comprises a sugar, an alcohol, or any combination thereof.

In a further embodiment, the hydrophilic carbon source comprises a carbon rich agricultural, industrial waste or residue, selected from the group comprising: cheese whey, wheat straw, sugar beet residues, sugar cane residues, crude glycerol, or any combination thereof.

In a further embodiment, the hydrophobic carbon source comprises an oil rich in triacylglycerols, selected from the group comprising: soybean oil, rapeseed oil, waste cooking fried oil, animal fat, algae driven oil.

In a further embodiment, the hydrophobic carbon source comprises free fatty acids, monoacylglycerol, diacylglycerol, or any combination thereof.

In a further embodiment, the hydrophobic carbon source is rich in free fatty acids and/or mono/di acylglycerol mixtures obtained without dedicated pre-treatment (e.g. olive pomace oils) or obtained from pre-treatment of the triacylglycerol rich substrates after their hydrolyses or transesterification.

In a further embodiment, the use of hydrophobic carbon source rich in free fatty acids and/or mono/di acylglycerol mixtures enhances the speed of production of MEL rich particles by at least 2 days.

In a further embodiment, the hydrophobic carbon source is added in step a), or up to 6 days after step a).

In a further embodiment, the hydrophobic carbon source and the hydrophilic carbon source are present in a ratio ranging from 10:1 to 1:1 by weight, respectively.

In a further embodiment, the solution in step b) comprises between 4 and 150 (g/L) of the MELs.

In a further embodiment, the process further comprises a step of removal of triacylglicerols by precipitation in methanol, while the MELs remains soluble.

In a further embodiment, the process further comprises a step of nanofiltering the MELs solution.

In a further embodiment, the MELs has a purity of more than 90%.

In another aspect of the disclosure, there is provided a composition comprising a plurality of mannosylerythritol lipids (MELs), wherein the MELs are in the form of particles.

In another aspect of the disclosure, there is provided an use of the device of the present disclosure, for production and/or purification of mannosylerythritol lipids (MELs).

In another aspect of the disclosure, there is provided an use of the present disclosure, for production and/or purification of mannosylerythritol lipids (MELs).

In another aspect of the disclosure, there is provided an use of the process of the present disclosure, for production and purification of mannosylerythritol lipids (MELs).

In another aspect of the disclosure, there is provided an use of the composition of the present disclosure, in agriculture, food, beverages, cleaning products, bioremediation, oil and gas industry, bio and fine chemical industry, cosmetics, pharmaceutical industry, or any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a block diagram of a MELs production system with integrated non-invasive product separation device (1) (also referred solely as device), and integrated pipeline for the circulation of fluids between bioreactor (2) and device (1), as well as solvent/warm water circulation between device (1) and reservoir (5).

FIG. 2 is a bar graph of the concentration of MELs present in extracts of MELs-rich beads and bead-free fermentation broth, after cultivation of M. antarcticus in 40 g/L of D-glucose and feeding 20 g/L of soybean oil (SBO), free fatty acid (FFA) or fatty acid methyl esters (FAME) at day 4, at 27° C., 250 rpm, for 7 days.

FIG. 3 is a diagram of the size and color of MELs-rich beads obtained at different fermentation periods in flasks fed with various lipid-based substrates at different fermentation days.

Graphic subtitle:

| Circle size | Diameter of MELs-rich beads | |
|---|---|---|
| Tiny | <1 | mm |
| Small | 1-3 | mm |
| Medium | 3-10 | mm |
| Large | >10 | mm |

Color code:

| | Color of MELs-rich beads for each |
|---|---|
| Circle color/pattern | fermentation day |
| white | green |
| White with black dots | yellow |
| Black with vertical | Light orange |
| white lines | |
| Black with horizontal | Dark Orange or brown |
| white lines | |

FIG. 4 is a bar graph of total MELs and lipids concentration, in beads and in fermentation broth, in flasks A (with glucose used for inoculum preparation) and B (with glycerol used for inoculum preparation), at the end of the 12-day fermentation. Main fermentation used as carbon source an initial concentration of 40 g/L of D-glucose and a mixture of 30 g/L of D-glucose and 40 g/L of colza oil added on day 4.

FIG. 5 is a diagram of the size and color of MELs-rich beads obtained at different fermentation periods in flasks fed with various substrates.

| Circle size | MELs-rich beads diameter | |
|---|---|---|
| Tiny | <1 | mm |
| Small | 1-3 | mm |
| Medium | 3-10 | mm |
| Large | >10 | mm |

| Circle color/pattern | Color of MELs-reach beads |
|---|---|
| white | green |
| White with checkered | Dark green |
| pattern | |
| White with black dots | yellow |
| Black with vertical | Light orange |
| white lines | |
| Black with horizontal | Dark Orange or brown |
| white lines | |

Color/pattern of the circle indicates the color of the MELs-rich beads for each fermentation day. 20 g/L of D-glucose was fed on day 4.

FIG. 6 is a picture of an exemplary design of the non-invasive product separation device (1). Left: Design of the MELs-rich bead separation vessel (Top cover removed for visibility of interior). Right: Side projection of the device (1), indicating flow directions of broth through the device (1), comprising at least one inlet (22), a grid (20) separating two chambers and at least one outlet (24).

FIG. 7 is a section of the block diagram of a MELs production system that integrates bioreactor (2) and non-invasive product separation device (1) for MELs-production, representing the viable cell circuit (see FIG. 1 for a circuit including MELs retrieval) 1—device (for product separation); 2—bioreactor; 3,10—valves; 4—pump (peristaltic pump); and 6—first pathway (broth flow).

FIG. 8 is a graph of the nanofiltration performance after 2, 4 and 6 diavolumes using membranes prepared from 22, 24 and 26% polybenzimidazole (PBI) solutions. Initial MELs purity submitted to nanofiltration is 82%.

### DETAILED DESCRIPTION OF THE INVENTION

According to some embodiments, the present disclosure provides a device and system for macrofiltration. In some embodiments, the macrofiltration comprises mannosylerythritol lipids (MELs) recovery.

The present disclosure is based, in part, on the finding that the macrofiltration device and system according to the present disclosure, allows the recovery of the product [e.g. mannosylerythritol lipids (MELs)], without interruption of the fermentation. This characteristic is especially important to reduce adverse effects of high concentrations of MELs and substrates in the fermentation broth (such as reduction in the liquid/gaseous oxygen transfer coefficient, kLa, i.e., increasing resistance to mass transfer, due to low air/liquid interface and high/medium viscosity, with consequent oxygen limitations and reduction of homogeneity of the medium).

In some embodiments, the removal of MELs from the fermentation broth, increases the production of MELs.

According to some embodiments, the present disclosure provides a process for enhancing mannosylerythritol lipids (MELs) production.

The present disclosure is based, in part, on a process, according to the present disclosure, that allows reducing the total volume and the number of organic solvents used in comparison to conventional methods known in the art (such as liquid-liquid extraction of the whole fermentation broth with organic solvents).

The present disclosure is based, in part, on the finding that the use of substrates with a high proportion of lipidic derivatives enhances the formation of more stable particles under fermenting conditions. In particular, feeds of lipids which are partially hydrolysed (containing significant fractions of free fatty acids and monoacylglycerols) or trans-esterified into single chain alkyl esters promote faster and more efficient formation of MELs-rich particles with an increased quality in terms of physical characteristics (e.g., firmness, size, density) and purity.

As used herein, “mannosylerythritol lipids (MELs)” refers to a glycolipid class of biosurfactants produced by a variety yeast and fungal strains. MELs are biosurfactant containing 4-O-β-D-mannopyranosyl-meso-erythritol as the hydrophilic group and a fatty acid and/or an acetyl group as the hydrophobic moiety. There are several structural variants of the MELs. These variants arise due to the following reasons: 1) number and position of the acetyl group on mannose or erythritol or both; 2) number of acylation in mannose; 3) fatty acid chain length and their saturation. The acyl groups in MELs may range from C7 to C14 and their proportions vary depending upon the carbon source used.

According to some embodiments, the present disclosure provides a device (1) for macrofiltration. In some embodiments, the device comprises a grid (20), the grid comprising a plurality of channels, each channel having a width between 0.3 mm and 10 mm. In some embodiments, each channel has a width between 0.3 mm and 10 mm, between 0.4 mm and 10 mm, between 0.5 mm and 10 mm, between 0.6 mm and 10 mm, between 0.7 mm and 10 mm, between 0.8 mm and 10 mm, between 0.3 mm and 8 mm, between 0.4 mm and 8 mm, between 0.5 mm and 8 mm, between 0.6 mm and 8 mm, between 0.7 mm and 8 mm, between 0.8 mm and 8 mm, between 0.3 mm and 5 mm, between 0.4 mm and 5 mm, between 0.5 mm and 5 mm, between 0.6 mm and 5 mm, between 0.7 mm and 5 mm, between 0.8 mm and 5 mm, between 0.3 mm and 2 mm, between 0.4 mm and 2 mm, between 0.5 mm and 2 mm, between 0.6 mm and 2 mm, between 0.7 mm and 2 mm, between 0.8 mm and 2 mm, between 0.3 mm and 1 mm, between 0.4 mm and 1 mm, between 0.5 mm and 1 mm, between 0.6 mm and 1 mm, between 0.7 mm and 1 mm, or between 0.8 mm and 1 mm, including any range therebetween. Each possibility represents a separate embodiment of the present disclosure. According to the present invention, the distance between the channels (width) is selected to be big enough to present no substantial shear stress on cells from a fermentation solution but small enough to retain MELs-rich beads within the device.

In some embodiments, the device (1) comprises a grid (20), the grid separating a first chamber and a second chamber each chamber positioned on an opposite side of the grid (20) along its longitudinal axis; at least one inlet (22) and at least one outlet (24), wherein: the at least one inlet (22) is configured to evenly supply a fluid to the first chamber; the second chamber is configured to collect a filtrated fluid; and the at least one outlet (24) is configured to connect the second chamber to a bioreactor (2).

In some embodiments, macrofiltration comprises mannosylerythritol lipids (MELs) recovery. In some embodiments MELs are in the form of particles. In some embodiments, the particles are retained in the first chamber.

In some embodiments, a device according to the present invention, is configured to collect the product (MELs rich particles) in situ, while recycling the fermentation broth (fermentation solution) containing cells and immature MELs-rich particles back to a fermenter (e.g., a bioreactor) at high flows and residence times tending to zero, but without significant cell damage. In some embodiments, the cell death is less than 2%.

As used herein, the term “particle” refers to a small piece of matter to which can be ascribed several physical or chemical properties such as volume, density or mass. A particle can be generally shaped as a sphere, incomplete-sphere, a rod, a cylinder, a ribbon, a sponge, and any other shape, or can be in a form of a cluster of any of these shapes, or can comprise a mixture of one or more shapes. In some embodiments, a particle according to the present disclosure refers to a bead. According to the present invention, the terms “particle” and “bead” are equivalent and used interchangeably.

In some embodiments, the size of the particles described herein represents an average or median size of a plurality of particles.

As used herein the terms “average” or “median” size refer to a diameter of the particles.

In some embodiments, the average or the median size of at least e.g., 10%, 20%, 30%, 40%, 50%, 60%, 70%, 80%, 90%, or 95% of the particles, is: between 1 mm and 50 mm, between 3 mm and 50 mm, between 5 mm and 50 mm, between 10 mm and 50 mm, between 15 mm and 50 mm, between 1 mm and 40 m, between 3 mm and 40 mm, between 5 mm and 40 mm, between 10 mm and 40 mm, or between 15 mm and 40 mm, including any range therebetween. Each possibility represents a separate embodiment of the present disclosure.

In some embodiments, a plurality of the particles has a uniform size, when present in the fermentation broth.

By “uniform” or “homogenous” it is meant to refer to size distribution that varies within a range of less than e.g., ±60%, ±50%, ±40%, ±30%, ±20%, or ±10%, including any value therebetween. Each possibility represents a separate embodiment of the present disclosure.

In some embodiments, a device according to the present invention is used for separating MELs particles from a fermentation solution. In some embodiments, the device retains the MELs particles within the device and the fermentation solution exits the device through the at least one outlet (24).

According to some embodiments, the present disclosure provides a use of the device described hereinabove, for production and/or purification of MELs.

According to some embodiments, the present disclosure provides a system for macrofiltration, comprising the device (1) of the present disclosure. In some embodiments, the system further comprises a bioreactor (2).

In some embodiments, the system comprises a first pathway (6), wherein the first pathway comprises a first valve (3), a first pump (4), the bioreactor (2) and a second valve (10), in fluid communication and subsequently positioned between the device (1) at least one outlet and inlet.

In some embodiments, the bioreactor (2) comprises a fermentation broth solution under conditions for the formation of MELs particles.

In some embodiments, the macrofiltration device and system are configured to work under sterile conditions, and without interruption of the fermentation and/or on an intermittent manner.

In some embodiments, the macrofiltration device retains the MELs particles within the device, while the filtrate (fermentation broth solution) is returned back to the bioreactor. In some embodiments, the system allows the continuity of fermentation, without substrate losses.

In some embodiments, the fermentation broth solution comprises a culture medium, nutrients, cells, and smaller poor quality (light, lipid richer, easily desegregated) MELs particles.

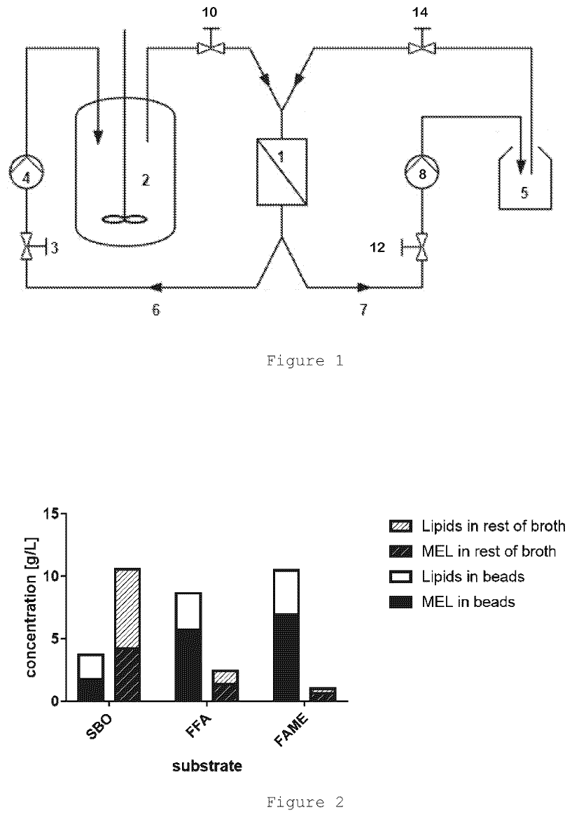

Reference is made to FIG. 1, that illustrates a schematic representation of a system according to the present disclosure. In some embodiments, the device (1) is used for the recovery of MELs-rich particles and is connected to the bioreactor (2) by a closed tube system (first pathway 6). In some embodiments, the system comprises a pump (4), to circulate the fermentation broth containing the beads rich in MELs through the device (1), which retains the beads. The fermentation broth, depleted of beads, is then returned to the bioreactor (2), in which the fermentation can continue and/or more substrate can be added for bioconversion.

According to some embodiments, the present disclosure provides a system for macrofiltration, comprising the device (1) of the present disclosure. In some embodiments, the system further comprises a reservoir (5).

In some embodiments, the system comprises an integrated product separation, through reservoir (5).

In some embodiments, the system comprises a second pathway (7), wherein the second pathway comprises a third valve (12), a second pump (8), the reservoir (5) and a fourth valve (14), in fluid communication and subsequently positioned between the device (1) outlet and inlet.

In some embodiments, device (1) is connected to a separate closed system (second pathway 7) connected to a reservoir (5). In some embodiments, the reservoir (5) comprises a solubilizing agent which can be recirculated through the device (1) to dissolve and recover the product beads. The flows of broth and solubilizing agent through the system are regulated by valves (3, 10, 12 and 14).

According to some embodiments, the present disclosure provides a use of the system described hereinabove for production and/or purification of (MELs).

According to some embodiments, the present disclosure provides a process for enhancing mannosylerythritol lipids (MELs) production. In some embodiments, the process comprises the steps of: a) contacting between 1% and 70% weight per weight (w/w) of a substrate and between 0.01% and 10% of a yeast in a culture medium solution under fermentation conditions thereby producing MELs in the fermentation broth solution; b) separating the MELs from the fermentation broth solution, and c) washing the MELs with a second solution, wherein the ratio of the second solution to the fermentation broth solution is between 3:10 to 1:50 volume per volume (v/v), thereby enhancing mannosylerythritol lipids (MELs) production.

In some embodiments, the process comprises contacting between 1% and 70%, between 2% and 70%, between 5% and 70%, between 10% and 70%, between 15% and 70%, between 20% and 70%, between 30% and 70%, between 1% and 50%, between 2% and 50%, between 5% and 50%, between 10% and 50%, between 15% and 50%, between 20% and 50%, between 30% and 50%, between 1% and 40%, between 2% and 40%, between 5% and 40%, between 10% and 40%, between 15% and 40%, or between 20% and 40%, weight per weight (w/w) of a substrate, including any range therebetween and between 0.01% and 10%, between 0.05% and 10%, between 0.09% and 10%, between 0.1% and 10%, between 0.5% and 10%, between 1% and 10%, between 2% and 10% between 0.01% and 5%, between 0.05% and 5%, between 0.09% and 5%, between 0.1% and 5%, between 0.5% and 5%, or between 1% and 5%, of a yeast in a culture medium solution, including any range therebetween, under fermentation conditions thereby producing MELs in the fermentation broth solution. Each possibility represents a separate embodiment of the present disclosure.

In some embodiments, the basal culture medium comprises NaNO₃, KH₂PO₄, MgSO₄·7H₃O, and a yeast extract, other complex supplement, or a defined biochemical supplement. In some embodiments, the fermentation broth solution comprises M. bullatus or M. antarcticus strains.

In some embodiments, the process comprises at step c) of washing the MELs with a second solution, wherein the ratio of the second solution to the fermentation broth solution is between 3:10 and 1:50 (v/v), between 3:10 and 1:40 (v/v), between 3:10 and 1:30 (v/v) or between 3:10 and 1:20 (v/v), including any range therebetween, thereby enhancing mannosylerythritol lipids (MELs) production. Each possibility represents a separate embodiment of the present disclosure.

In some embodiments, the second solution comprises an alcoholic solution and/or an aqueous solution. In some embodiments, the second solution comprises methanol.

In some embodiments, the recovery process is performed in the device or in the system described hereinabove.

In some embodiments, step b) separating the MELs from fermentation broth solution is performed in the device (1). In some embodiments, step c) washing the MELs with a second solution, wherein the ratio of the second solution to the fermentation broth solution is between 3:10 to 1:50 volume per volume (v/v), thereby enhancing mannosylerythritol lipids (MELs) recovery process, is performed in the device (1).

In some embodiments, step c) washing the MELs with a second solution, wherein the ratio of the second solution to the fermentation broth solution is between 3:10 to 1:50 volume per volume (v/V), thereby enhancing mannosylerythritol lipids (MELs) recovery process, is performed in the device (1), in the reservoir (5), or both

In some embodiments, the a) contacting between 1% and 70% weight per weight (w/w) of a substrate and between 0.01% and 10% of a yeast in a culture medium solution under fermentation conditions thereby producing MELs in the fermentation broth solution, is performed in the bioreactor (2), in the device (1), or both.

In some embodiments, process further comprises a step of circulating the fermentation broth solution from step b), back to step a).

In some embodiments, the MELs has a purity of more than 55%, more than 60%, more than 65%, more than 69%, more than 70% or more than 75% (w/w), including any value therebetween. Each possibility represents a separate embodiment of the present disclosure.

In some embodiments, the MELs is in the form of particles.

In some embodiments, the particles have a particle size of more than 1 mm, more than 2 mm, more than 3 mm, more than 5 mm, or more than 10 mm, including any value therebetween. Each possibility represents a separate embodiment of the present disclosure.

In some embodiments, the particles have a particle size between 1 mm and 50 mm, between 3 mm and 50 mm, between 5 mm and 50 mm, between 10 mm and 50 mm, between 15 mm and 50 mm, between 1 mm and 40 m, between 3 mm and 40 mm, between 5 mm and 40 mm, between 10 mm and 40 mm, or between 15 mm and 40 mm, including any range therebetween. Each possibility represents a separate embodiment of the present disclosure.

In some embodiments, the substrate comprises a hydrophilic carbon source, hydrophobic carbon source, or both.

In some embodiments, the hydrophilic carbon source comprises an alcohol, a sugar, or any combination thereof. Non-limiting examples of alcohols according to the present invention include glycerol, erythritol, methanol, ethanol, propanol, butanol, pentanol, hexanol, isobutanol, and combinations thereof. Non-limiting examples of sugars (carbohydrates) according to the present invention include glucose, xylose, saccharose, lactose, galactose, sucrose, fructose, trehalose, mannose, mannitol, and/or maltose, and combinations thereof.

In a further embodiment, the hydrophilic carbon source comprises a carbon rich agricultural or industrial waste or residue, such as cheese whey, wheat straw, sugar beet residues, sugar cane residues and crude glycerol.

In some embodiments, the hydrophobic carbon source comprises an oil rich in triacylglycerol. Non-limiting examples of hydrophobic carbon source according to the present invention include soybean oil, rapeseed oil, coconut oil, canola oil, safflower oil, rice bran oil, olive oil, corn oil, sesame oil, linseed oil, waste cooking oil, cooking oil residues, animal fat, algae driven oil, and combinations thereof.

In some embodiments, the hydrophobic carbon source comprises free fatty acids, monoacylglycerol, diacylglycerol, or any combination thereof.

The present invention is based in part, on using substrates comprising a high proportion of lipidic derivatives thus enhancing the formation of more stable particles under fermenting conditions. In particular, feeds of lipids which are partially hydrolysed (containing significant fractions of free fatty acids and monoacylglycerols) or trans-esterified into single chain alkyl esters promote faster and more efficient formation of particles with an increased quality in terms of physical characteristics (e.g., firmness, size, density) and purity. The use of such substrates enables more efficient (e.g. faster and with increased quality) particle (bead) production. Without being bound to any particular theory, the presence of triacylglycerols, when in significant amounts, might inhibit quality and particle formation. Therefore, using strategies where triacylglycerols are absent, in low amounts or are broken down early on in the fermentation, allows the production of higher quality particles made of MELs and presence of smaller amount of lipid derivatives on the fermentation broth.

In some embodiments, the hydrophobic carbon source comprises (i) triacylglycerol rich vegetable oil feeds that have been submitted to a pre-treatment step, (e.g., using lipases, similar enzymes or chemical catalysis), to hydrolyse triacylglycerols into monoacylglycerols and free fatty acids or, in the presence of an alcohol, trans-esterified them into single chain alkyl esters, (ii) lipids feeds, such as olive pomace oils or biodiesel production waste streams (such as crude glycerol rich in free fatty acids and monoacylglycerols), which by their nature have a compositions with lower triacylglycerols content and rich on free fatty acids or monoacylglycerols, or any combination of (i) and (ii).

In some embodiments, the hydrolysis of the hydrophobic carbon source (e.g. vegetable oil) is done by mixing lipase-rich supernatant with 80 g/L of vegetable oil, and incubated at 27° C. for 48 h. For the generation of methyl esters, the vegetable oil is mixed with methanol in a 1:4 molar ratio (vegetable oil/methanol) and incubated in a similar fashion. In some embodiments, commercially available immobilized CAL-B (lipase B of Candida antarctica) was used instead.

In some embodiments, the hydrophobic carbon source comprises less than 60% in triacylglycerols, less than 50% in triacylglycerols, less than 40% in triacylglycerols, less than 30% in triacylglycerols, or less than 20% in triacylglycerols, including any value therebetween. Each possibility represents a separate embodiment of the present disclosure.

In some embodiments, the use of substrates comprising a high proportion of lipidic derivatives enhances and advances the formation of more stable particles under fermenting conditions by at least 1 day, at least 2 days, at least 3 days, at least 4 days, or at least 5 days. Each possibility represents a separate embodiment of the present disclosure.

In some embodiments, the use of hydrophobic carbon source rich in free fatty acids and/or mono/di acylglycerol mixtures enhances the production of MELs rich particles by at least 2 days, at least 3 days, at least 4 days, or at least 5 days. Each possibility represents a separate embodiment of the present disclosure. In some embodiments, enhancing refers to time reduction on the formation of MELs and/or increased quality in the MELs formed.

In some embodiments, the hydrophobic carbon source is added in step a), or up to 6 days after step a).

In some embodiments, the hydrophobic carbon source and the hydrophilic carbon source are present in a ratio ranging from 10:1 to 1:1, 9:1 to 1:1, 8:1 to 1:1, 7:1 to 1:1, 6:1 to 1:1, 5:1 to 1:1, 10:1 to 9:1, 9:1 to 6:1, 8:1 to 5:1, 7:1 to 5:1, 6:1 to 2:1, or 5:1 to 1:1, by weight, respectively, including any range therebetween. Each possibility represents a separate embodiment of the present disclosure.

In some embodiments, the solution in step b) comprises from 4 to 150 (g/L), 5 to 150 (g/L),, 10 to 150 (g/L), 20 to 150 (g/L), 40 to 150 (g/L), 50 to 150 (g/L), 100 to 150 (g/L), 4 to 120 (g/L), 5 to 120 (g/L), 10 to 120 (g/L), 20 to 120 (g/L), 40 to 120 (g/L), 50 to 120 (g/L), 100 to 120 (g/L), 4 to 100 (g/L), 5 to 100 (g/L),, 10 to 100 (g/L), 20 to 100 (g/L), 40 to 100 (g/L), 50 to 100 (g/L), or 100 to 150 (g/L), of the MELs, including any range therebetween. Each possibility represents a separate embodiment of the present disclosure.

In some embodiments, the process further comprises a step of removal of tryacylglicerides by their precipitation in methanol. In some embodiments, the MELs remains soluble in the solution. In some embodiments, the step of removal of tryacylglicerides is repeated at least 1 time, at least 2 times, at least 3 times at least 4 times, or at least 5 times. Each possibility represents a separate embodiment of the present disclosure.

In some embodiments, the step of removal of triacylglicerides by their precipitation in methanol, enables the removal of any residual triacylglycerols present in the particles. In some embodiments, the content of tryacylglicerides in the crude MELs is less than 45%, 40%, 30%, 20%, 10%, less than 7%, or less than 5%. Each possibility represents a separate embodiment of the present disclosure.

In some embodiments, the process further comprises a step of nanofiltering the MELs solution. In some embodiments, the MELs particles after nanofiltration have a purity of more than 90%, more than 92%, more than 95%, more than 96%, or more than 98%, including any value therebetween. Each possibility represents a separate embodiment of the present disclosure. In some embodiments, the nanofiltration step enables the removal of any residual smaller lipidic contaminants. In some embodiments, the process further comprises a step of evaporating the solution.

In some embodiments, nanofiltration refers to diafiltration.

In some embodiments, the step of nanofiltering comprises at least 2 diavolume (DV), at least 3 DV, at least 4 DV, at least 5 DV, or at least 6 DV. Each possibility represents a separate embodiment of the present disclosure.

As used herein, one “diavolume (DV)” refers to the volume amount of fresh solvent (e.g. methanol) that equals the volume of the initial solution submitted to a diafiltration.

In some embodiments, according to the present invention, the removed lipidic contaminants can be reused as substrate for subsequent fermentations after the retrieval of most of the methanol. In some embodiments, the present invention is based in part, on using small quantities of methanol thus preventing the inhibitory effects on the microorganisms.

According to some embodiments, the present disclosure provides a use of the process described hereinabove, for production and purification of mannosylerythritol lipids (MELs).

According to some embodiments, the present disclosure provides a composition comprising a plurality of mannosylerythritol lipids (MELs), wherein the MELs is in the form of particles.

According to some embodiments, the present disclosure provides a use of the composition disclosed hereinabove, in agriculture, food, beverages, cleaning products, bioremediation, oil and gas industry, bio and fine chemical industry, cosmetics, pharmaceutical industry, or any combination thereof.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

As used herein, the singular form “a”, “an” and “the” include plural references unless the context clearly dictates otherwise. For example, the term “a valve” or “at least one valve” may include a plurality of valves, including mixtures thereof.

Throughout this disclosure, various embodiments disclosed may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number (i.e. ranges between 2 and 4 or ranges from 2 to 4) are used herein interchangeably and are meant to include the first and second indicated numbers and all the fractional and integral numerals therebetween.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub combination or as suitable in any other described embodiment of the disclosure. Certain features described in the context of various embodiments are not to be considered essential features of those embodiments, unless the embodiment is inoperative without those elements.

### EXAMPLES

Reference is now made to the following examples, which together with the above descriptions illustrate some embodiments of the invention in a non-limiting fashion.

### Example 1

### Preliminary Experiments

### MELs-Rich Beads Formation.

A method according to the present disclosure includes a stage of fermentation with a first step to achieve the phase of cell growth and a second step that comprises a stationary phase characterized by high MELs production. For this fermentation, hydrophilic substrates (e.g. D-glucose, D-xylose, glycerol) can be used, along with hydrophobic substrates (vegetable oils, acyl glycerides, alkanes, methyl esters, carboxylic acids), or their mixture. While formation of MELs-rich beads is described in literature for fermentations in shake-flask; the appearance of such beads is intermittent or inexistent in fermentations taking place on bioreactor, most probably as a consequence of the use of high agitation and aeration in the cell growth and MELs production steps.

Soybean oil, a vegetable oil rich in triacylglycerols, is according to the literature the reference substrate used for MELs production. Therefore, following the best practices described in the literature to produce large amounts of MELs in bioreactor, soybean oil (SBO) was the substrate initially used. This substrate was added as received at values 40 g/L at day 0 and 40 g/L at day 4; such concentrations are calculated on the basis of weight of substrate added to total volume of fermentation broth. The production of MELs-rich beads was only observed after 9 days, and the accumulated substrate resulted on increase on substrate concentrations to values above 40 g/L. Still, the produced beads were of lower quality (i.e., soft, gelatinous, of low density, easy to disaggregate, with high lipid content, rich in biomass and water, and easy to break under agitation). Such beads could not be easily recovered using macrofiltration.

The initial approach to improve MELs productivity and purity was to reduce vegetable oil fed as substrate and to complement initial carbon source feed with a hydrophilic substrate, such as D-glucose, D-xylose or glycerol. Several substrates were used with the aim to improve MELs productivities, with various results, but without significant improvements in terms of speed on beads formation or their quality. In a separate study, the inventors did hypothesize that the metabolic limitation to MELs synthesis from triacylglycerol rich substrates was due to slow yeast lipase catalytical activity. Therefore, the inventors performed yeast fermentations for MELs production with lipidic feeds which were partially hydrolysed, i.e., rich in free fatty acids and monoacylglycerols. Surprisingly, when such partially hydrolysed lipids were used in smaller feed amounts, in comparison to the amounts of previously used vegetable oils, they enable better bead formation.

The same improvement on beads quality and formation speed can be obtained using (i) triacylglycerol rich vegetable oil feeds that had been submitted to a pre-treatment step, using lipases, similar enzymes or chemical catalysis, to hydrolyse triacylglycerols into monoacylglycerols and free fatty acids or, in the presence of an alcohol, trans-esterified them into single chain alkyl esters, or (ii) lipids feeds, such as olive oil pomace or biodiesel production waste streams (such as crude glycerol rich in free fatty acids and monoacylglycerols), which by their nature have a compositions with lower triacylglycerols content and rich on free fatty acids or monoacylglycerols. In the current document, the inventors designate the above mentioned free fatty acids and monoacylglycerols or single chain alkyl esters, that are obtained from triacylglycerol as lipid derivatives.

Therefore, the current invention discloses that the use of substrates with a high proportion of lipidic derivatives enhances the formation of more stable product beads under fermenting conditions. In particular, feeds of lipids which are partially hydrolysed (containing significant fractions of free fatty acids and monoacylglycerols) or trans-esterified into single chain alkyl esters promote faster and more efficient formation of beads with an increased quality in terms of physical characteristics (firmness, size, density) and purity. The use of such substrates enables more efficient bead production, probably because the presence of triacylglycerols, when in significant amounts, inhibits quality bead formation. Therefore, using strategies where triacylglycerols are absent, in low amounts or are broken down early on in the fermentation, allows the production of higher quality beads made of MELs and presence of smaller amount of lipid derivatives on the fermentation broth.

Several distinct characteristics of MELs-rich beads can be used as indicators of their quality, which mainly relates to the content of MELs within the beads. Buoyancy of the beads is related to the content of triacylglycerols. Beads with a non-negligible concentration of triacylglycerol float on the non-agitated fermentation broth, while the beads which do not contain triacylglycerols tend to not float. Still, MELs-rich beads sedimentation does not occur at a level which could be used to efficiently recover them from the fermentation broth, as beads coalescence into a separate cohesive is not formed. Moreover, triacylglycerol presence in the beads makes them soft and gelatinous and prevents their recovery using macrofiltration or other physical methods. On the other hand, beads with higher levels of MELs (>55%) and insignificant amounts of triacylglycerols (<5%) become firm, enabling efficient recovery. Beads size depends on mixing regime and system (e.g., the beads are larger when orbital shaker is used for shake flasks and smaller when impeller is used for bioreactor fermentations). Still size of beads is another indicator of bead quality. In shake flasks, MELs-rich beads of relative diameter <1 mm had <40% purity, those ranging from 1-3 mm had 40-50% purity, those with a relative diameter of 3-10 mm had 50-60% purity, while beads of purity higher than 60% had a diameter larger than 10 mm. Similar relative co-dependency of purity (in terms of MELs content) and size was determined for bioreactor fermentations, where a fermentation agitated by a Rushton impeller (at 400 rpm) generated beads of 1-3 mm, with a purity of 65-70%. Finally, coloration of the beads can be an indicator of their purity, whereas transparent beads with a relatively darker color have a higher purity, and beads containing larger fractions of lipids remain opaque and lighter in color. The coloration of the beads depends on the medium composition, mainly on the hydrophobic substrate used, and for vegetable oils ranges from yellowish green for impure beads (with 35-50% purity), to dark-orange and brown beads (with a purity >65%). In this document the inventors designate MELs-rich beads to be of good quality when those are firm, large, dense and robust.

Following the substrate feed strategies here disclosed allowed to form beads at lower MELs titers compared to feed strategies where normal unhydrolyzed lipid feeds are used. In one instance, the use of partially hydrolysed soybean oil enabled the formation of beads of good quality two days earlier in the fermentation than for the case when pure soybean oil was used, and enabled formation of beads in fermentations beads where MELs concentrations were so low as 5 g/L. This fact contradicts the previous state of the art, which relates bead appearance to specific MELs concentrations high thresholds. In this sense, partially hydrolysed oils, lipid feeds with a high degree of acidity, or agro-industrial residues with high levels of hydrolysed lipids can promote MELs-rich bead formation (e.g., the inventors confirmed that this is the case for olive pomace oil and low-quality olive oil). The same observation is valid for industrial residues rich on trans esterified lipids such as the ones obtained from biodiesel production.

Another aspect beneficial to improve MELs-rich beads formation is disclosed. The rational to use two carbon sources was to facilitate, at first place, biomass growth and enzymes production using a readily metabolizable hydrophilic substrate, such as D-glucose; and then, to introduce the hydrophobic substrate, vegetable oils or its lipid derivatives, to boost the formation of MELs, a secondary metabolite. However, surprisingly, the when protocol was not followed and the two carbon sources were added together at the beginning of the fermentation, the MELs-rich beads appear faster. This was quite unexpected as usually is assumed that it is beneficial a stepwise approach to avoid eventual catabolic repression when using two substrates simultaneously.

Finally, when glycerol, which is usually used as the cryopreserving for storage of the yeast cultures, was used during the inoculum preparation stage as the carbon source, it enabled the production of beads of better quality and in higher amounts (relative to the total amount of MELs present in the broth), independently of the hydrophilic carbon source used in the main fermentation.

### Downstream Route: Turning Around the Filtration Rational.

Typically, when combining filtrations with fermentations, cells are retained by the filter and the product is collected on the filtrate. One of the disadvantages of such configurations is the potential washout of unconsumed substrates, which need to be managed using low permeate flowrates (i.e., large residential time). When such configuration was initially assessed by our team, with an ultra and microfiltration membrane, surprisingly we verified that the concentration of MELs on the filtrate was very low. Then with the emergence of large, dense and robust MELs-rich beads, using the conventional membrane configuration the product was definitely retained by the membranes together with the cells, turning coupling of fermentation and membranes useless. In a failed experiment, due to pin holes on the membrane filter, it was observed beads retention, while cells were leakage to the filtrate.

The observations described above lead to the current invention, in which the presence of high-quality beads (firm, large, dense and robust) enables a non-obvious and new design in which a macro-filtration is used under sterile conditions, in situ, without interruption of the fermentation and on an intermittent manner. In such operation, instead of cells, the macrofiltration separation retains the large MELs-rich beads within the device (1), while the filtrate, comprised by the culture medium, nutrients, cells, and smaller poor quality beads (lighter, lipid richer, easily desegregated), is returned back to the bioreactor, allowing the continuity of fermentation, without substrate losses. In general, beads which are soft, small, and are unable to be retained on the macrofiltration grid, within the device (1), are considered being of poor quality and have a MELs content of <55% and size of <3 mm or 1 mm, respectively for MELs particles produced at shake flasks and stirrer bioreactor. This configuration allows to operate the system in a closed system, and importantly, to use residential time tending to zero, i.e. a fast recirculating flowrate. However, considering shear stress effects on the cells, the design of the macrofiltration separator and selection of flowrates was not trivial. A modified crossflow setup was selected, and the tube size and distance between the grids were selected to be big enough to prevent any relevant shear stress on the cells and broth retention within the device.

This coupled system can be used continuously or discontinuously, being triggered by the presence of product beads in the fermentation broth in the bioreactor. This configuration allows that after removing the beads from the fermentation broth, the coupled system is turned off, and new substrate can be added into the bioreactor. This procedure prevents contamination of the product with substrate, which makes it difficult to further purify.

The MELs accumulated in the device can be removed using a solubilizing agent which removes it from the system (e.g., alcoholic solvent). Alternatively, the product accumulated in the device can be retrieved by mechanical removal of the beads retained in the macrofiltration device, or by washing with heated water, with a solvent-free product being acquired. The device design is presented in the examples section.

### Downstream Route: From Multiple Solvents and Several Extraction Steps to the Use of a Single Solvent on Small Amounts.

Conventionally, MELs downstream route starts with organic solvent extraction of the complete fermentation broth, using multiple extraction steps and high volumes of ethyl acetate or ethyl-tert-butyl ether. For one liter of fermentation broth, 2-3 liters of organic solvent (volumetric ratio of 2:1 to 3:1 solvent to fermentation broth) are required on the initial downstream step. However, the use of the device (1) allows to recover the MELs from the fermentation broth using a small amount of solubilizing agent (between 100 ml to 1 L and 100 mb to 5 L i.e., proportion of solvent to the fermentation broth from 1:10 to 1:50, respectively). In addition, the use of the proposed method reduces the energy required for solvent removal. Moreover, on the conventional downstream route, which the first stage is an organic solvent/aqueous liquid-liquid extraction, the selection of the extracting organic solvent is based on their immiscibility with water. This limits the possibility of using polar solvents in downstream.

Most of the non-polar solvents are non-sustainable and of petrochemical origin (hexane, chloroform, etc.). Therefore, in conventional MELs downstream routes, further steps aiming at MELs purification that require the use of polar solvents. But using water miscible polar solvents (e.g., alcoholic solvents) is only possible in combination with non-polar solvent, in two-phase solvent extraction, or after solvent swap of crude MELs, by evaporation of the non-polar solvent initially used on the extraction step, and further dilution of the crude MELs in the polar solvent (process that is solvent and energy consuming and that can damage the product). In our novel system, the MELs-rich beads are retained in the separation vessel together with a very small fraction of water, negligible in comparison to the water volume in the fermentation broth. Therefore, MELs can be transferred directly from the device to a wide range of solvents, including sustainable polar solvents, such as alcohols. This implies a surprising ability to use solvents miscible with water for the extraction of the MELs from the fermentation broth, which would be impossible in a normal process setup. Alternatively, there is a possibility of obtaining a solvent free product, with the use of heated water for washing out the beads collected in the device (1).

### Downstream Route: the Surprising Benefits of Using Methanol on Downstream Processing.

In some cases, when using the new non-invasive product separation device (1) to recover MELs, the inventors did observe an awkward phenomenon in MELs separation processes from fermentations with a higher content of unconsumed lipidic substrates, rich in triacylglycerols, present. On such assays, when methanol was used for the removal of the MELs-rich beads from the separation vessel (1) and the resulting solution was not straightforward processed, but was left overnight, the inventors were surprised to observe a layer to be formed in the bottom of the vessel. Further investigation of this bottom layer and of the top methanol liquid phase allowed the inventors to establish that the triacylglycerols were being separated in the bottom phase, but virtually all the MELs remains on the top phase dissolved in methanol. Then the inventors established that this step can be repeated multiple times and enables the removal of triacylglycerols until their content in the crude MELs to be lower than 5%. This step enables the removal of any residual triacylglycerols present in the beads.

While the previous findings allowed to remove the triacylglycerols, the removal of any monoacylglycerols, free fatty acids or single chain alkyl esters was achieved by submitting the methanol solution of crude MELs directly to nanofiltration step, with the different MELs isoforms to be retained and the single chain alkyl esters or free fatty acids and monoacylglycerols filtrated through the membrane. The permeate solution can be concentrated with recovery of the methanol for further used and the non-distillated bottom phase, rich in substrate, recycled back to the fermentation. A surprising element to this is that any methanol present in the system, that finds its way to the bioreactor during subsequent bead collections, does not compromise the fermentation. Namely, our experience shows that the methanol, such as the one released by the hydrolysis of the methyl esters, disappears from the fermentation broth, probably due to being metabolized by the cells.

### Example 2

### Production of MELs With Formation of MELs-Rich Beads

### 1.A. Pre-Culture for Cell Growth

### Culture Medium With Carbon Source:

D-glucose, 40 g/L;
NaNO₃, 3 g/L;
KH₂PO₄, 0.3g/L;
MgSO₄·7H₂O, 0.3 g/L;
Yeast extract, 1.0 g/L;

All compounds were prepared in concentrated solutions, which were autoclaved at 121° C. for 20 minutes. After cooling, those solutions and sterile distilled water were added together in a sterile Erlenmeyer flask to obtain the concentrations described above, under sterile conditions. The medium is inoculated with M. antarcticus or M. bullatus, and incubated for 2 days under aerobic conditions and constant agitation at a fermentation temperature of 27° C.

### 1.B. Fermentation Process Lipase Production and Substrate Pre-Treatment

### Culture Medium With Carbon Source:

D-glucose, 40 g/L;
NaNO₃, 3 g/L;
KH₂PO₄, 0.3 g/L;
MgSO₄·7H₂O, 0.3 g/L;
Yeast extract, 1 g/L;

Cultivation conditions described here were

followed with the goal of producing lipases by M. antarcticus: The culture medium for the fermentation process was prepared in sterile water, with initial pH equal to 6. The culture medium was inoculated with 10% (v/v) of preculture prepared in 1.A. using M. antarcticus, and incubated in an Erlenmeyer flask under aerobic conditions and constant agitation at a temperature of 27° C. Concerning carbon source, this fermentation started using the 40 g/L of D-glucose present on the medium and, additionally 40 g/L of glucose was fed on day 4. After 7 days, the content of the flask was centrifuged for 8 min at 8000 rpm, and the lipase-rich supernatant was collected and used for hydrolysis of triacylglycerol rich vegetable oil.

The hydrolysis of the vegetable oil took place in a separate flask, where the lipase-rich supernatant is mixed with 80 g/L of vegetable oil, and incubated at 27° C. for 48 h.

For the generation of methyl esters, the vegetable oil was mixed with methanol in a 1:4 molar ratio (vegetable oil/methanol) and incubated in a similar fashion. However, instead of the supernatants, commercially available immobilized CAL-B was used.

The obtained lipid derivatives feeds comprised about 51.65% and 25.71% of the reaction products, respectively, free fatty acid (FFA) and fatty acid methyl esters (FAME). The compositions of these lipid derivative feeds are presented in Table 1. For the purpose of this disclosure a “FFA Enriched” or “FAME enriched” are substrates that contain less than 60% in triacylglycerols.

TABLE 1
Composition of the vegetable oil used as substrate
in M. antarcticus cultivations.

| Component (%) | SBO | FFA | FAME |
|---|---|---|---|
| Free fatty acids | 0.4 | 51.6 | 26.5 |
| Monoacylglycerols | 0.0 | 1.6 | 25.7 |
| Diacylglycerols | 0.6 | 13.7 | 6.3 |
| Triacylglycerols | 99.0 | 33.1 | 15.8 |
| Esters | / | / | 25.7 |

### 1.C. Detailed Example of Fermentation Processes for MELs-Rich Beads Production

### Culture Medium With Carbon Source:

Carbon source (variable)
KH₂PO₄, 0.3 g/L;
MgSO₄·7H₂O, 0.3 g/L;
Yeast extract, 1.0 g/L;

The culture medium for the fermentation process was prepared in sterile water, with initial pH equal to 6. The culture medium was inoculated with 10% (v/v) of preculture prepared in 1.A., and incubated under aerobic conditions and constant agitation at a temperature of 27° C.

As for the carbon sources used, this fermentation strategy used 40 g/L of D-glucose, a hydrophilic carbon source, present in the medium, to support the first phase of cell growth and adaptation. Additionally, a hydrophobic carbon source was fed on day 4. This source consisted of 20 g/L of vegetable oil used as received, or enzymatically pre-treated, to decrease their content on triacylglycerols below 50% and increase its content in (i) FFA or (ii) FAME.

The production of MELs was assessed after the addition of the hydrophobic substrate. MELs started to organize themselves in the fermentation broth in the form of firm and consistent MELs-rich beads. MELs-rich beads formation was used, for the purpose of this invention, as a measure of MELs production over the fermentation.

When unprocessed soybean oil, rich in unbroken triacylglycerols, is used, the formation of MELs-rich beads was a slow process, taking 7 days for the appearance of first beads, and 10 days for the beads to achieve sufficient quality. The MELs-rich beads vary in their composition and are present when MELs and lipids in the fermentation broth are in different ratios. The inventors verified that the MELs-rich beads are never robustly formed when there is a significant concentration of unhydrolyzed triacylglycerols in the broth.

Under the conditions where the lipid derivatives, obtained by pre-treating the vegetable oils to obtain (i) FFA enriched or (ii) FAME enriched, were fed as hydrophobic carbon sources, the MELs-rich beads formation took place earlier in the fermentation, with formation of higher quality and larger MELs-rich beads, obtained in higher numbers.

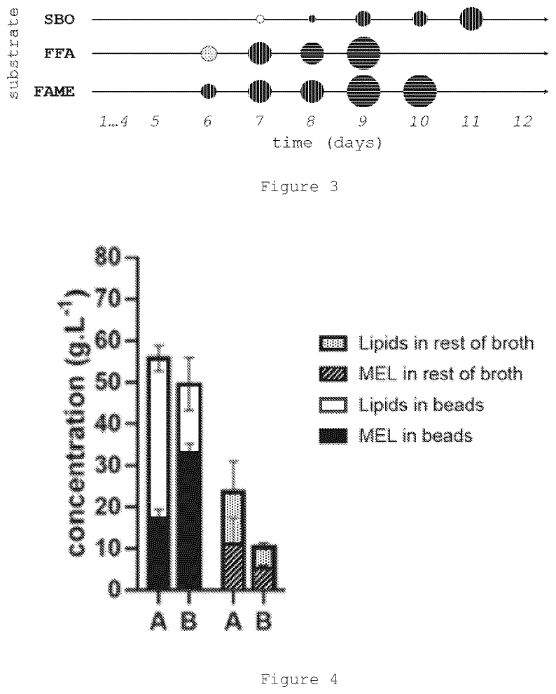

Regardless the type and quality of beads form, those are always difficult to isolate by sedimentation. It was observed that, although MELs-rich beads come with various purity (˜50-85%) and are formed when different ratios of MELs and lipids are present in the fermentation broth, they are never composed solely of MELs (FIG. 2). The inventors disclose that free fatty acids and monoacylglycerols play an important role in MELs-rich bead formation. This explains why the inventors observed that the MELs-rich beads formed tend to dissolve at later times of longer fermentations, as the small lipid derivates are metabolized by the yeast. This is counterintuitive, as the MELs-rich beads dissociate despite the concentration of MELs increasing with the fermentation progress. This further establishes the observation that lipid feeds rich in single chain free fatty acids and moncacylglycerols positively affect MELs-rich beads formation.

Besides the standard sampling for estimation of fermentation parameters (Biomass, MELs production and substrate consumption), the fermentation time at which the MELs-rich beads appeared was also registered, along with their characterization concerning physical properties-size and color. The size, and color of these beads were estimated and are presented in FIG. 3 for each fermentation time and conditions used.

In these experiments, the color of the beads provides an indication of how “mature” the beads are, i.e. how rich in MELs they are. Green and yellow beads tend to have lower MELs levels and to be richer, respectively, in triacylglycerols and free fatty acids, often lacking firmness; while darker orange beads are firm and have high MELs concentrations (>80%).

The use of the lipidic derivatives (i.e. FFA enriched or FAME enriched), a substrates, effectively reduced the time needed for the appearance of MELs-rich beads by several days, and positively affected such beads size and quality, as can be seen in FIG. 3.

### 1.D. MELs Production—Use of Glycerol as Inoculum Substrate for Promoting MELs-Rich Bead Formation.

Pre-fermentation (inoculum) culture medium with carbon source: D-Glucose or Glycerol, 40 g/L;
NaNO₃, 3 g/L;
KH₂PO₄, 0.3 g/L;
MgSO₄·7H₂O, 0.3 g/L;
Yeast extract, 1.0 g/L;

All compounds were prepared in concentrated solutions and autoclaved at 121° C. for 20 minutes. After cooling, those solutions and sterile distilled water were added together in sterile Erlenmeyer flask to obtain the concentrations described above, under sterile conditions. The medium was inoculated with M. antarcticus and incubated for 2 days under aerobic conditions and constant agitation at a fermentation temperature of 27° C. D-glucose and glycerol were used as carbon source, respectively, for the preparation of the inoculum used in conditions A and B of the main fermentations.

### Main Fermentation Culture Medium With Carbon Source:

Carbon source: D-Glucose+rapeseed oil
KH₂PO₄, 0.3 g/L;
MgSO₄·7H₂O, 0.3 g/L;
Yeast extract, 1.0 g/L;

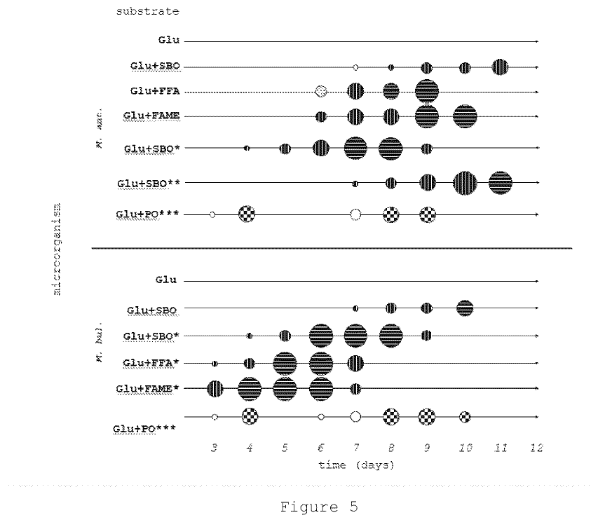

The culture medium for the fermentation process was prepared in sterile water, with initial pH equal to 6. The main fermentations were carried out in shake flasks and started by inoculating the culture medium with 10% (v/v) of a pre-culture prepared as described above, i.e. using D-glucose (condition A) or glycerol (condition B) as carbon source on the inoculum preparation stage of main fermentation carried out on condition A or B, respectively. The media and carbon source used on all the main fermentation conditions was similar. All the main fermentations started by using 40 g/L of D-glucose, present on the medium and, additionally, a mixture of 30 g/L of D-glucose and 40 g/L rapeseed oil was fed at day 4. The content of MELs and lipids in the beads, as well as in the beads-free fermentation broth are presented in FIG. 4.

The main fermentations, whose inoculum was prepared with glycerol produced more MELs and had fewer residual lipids in the end of the fermentation. In addition, glycerol used for inoculum preparation resulted in higher production of MELs-rich beads, which is an interesting opportunity for retrieving the surfactant from the broth more efficiently at the end of the fermentation.

### 1.E. Overview on Various Fermentation Processes and MELs-Rich Beads Production

### Culture Medium With Carbon Source:

Carbon source (variable)
KH₂PO₄, 0.3 g/L;
MgSO₄·7H₂O, 0.3 g/L;
Yeast extract, 1.0 g/L;

The culture medium for the fermentation process was prepared in sterile water, with initial pH equal to 6. The culture medium is inoculated with 10$ (v/v) of preculture prepared in 1.A. using M. antarcticus or M. bullatus, and incubated under aerobic conditions and constant orbital agitation at a temperature of 27° C.

As for the carbon sources used, this strategy used a hydrophilic carbon source and a hydrophobic carbon source. The hydrophobic source consisted of 20 g/L of triacylglycerol rich vegetable oil (e.g., soybean oil, rapeseed oil, waste cooking fried oil) fed to the fermentation as received, or enzymatically pre-treated to decrease its content on triacylglycerols below 50% and increase its content on (i) FFA or (ii) FAME. Moreover, food industrial residues, such as olive pomace oil were also used as lipidic derivatives. D-glucose can be used as hydrophilic carbon source. Moreover, other hydrophilic carbon can be used (other carbohydrates, glycerol, etc.), without negative impact on the production of MELs-rich beads during the fermentation; unlike the selection of the hydrophobic carbon source to be used on the main fermentation, which has a substantial impact on the formation of MELs-rich beads formation time and their quality. The hydrophilic and hydrophobic carbon sources can be added together at the start of the fermentation or/and stepwise. Table 2. Presents the MELs attained for different substrates combinations.

TABLE 2
Substrate feeding strategies (substrate type and
concentration and day of addition) for various fermentations
and maximum MELs concentrations attained for M. antarcticus
and M. bullatus.

| | | Substrate | | Maximum |
|---|---|---|---|---|
| | | Added | Day of | MELs |
| Fermentation | Microorganism | (g/L) | addition | (g/L) |
| Glucose | M. antarcticus | 40 | 0 | 5.40 |
| | M. bullatus | 40 | 0 | 3.40 |
| Glucose | M. antarcticus | 40 | 0 | 11.64 |
| Soybean oil | | 20 | 4 | |
| (SBO) | | 40 | 0 | 9.84 |
| | | 20 | 0 | |
| | M. bullatus | 40 | 0 | 10.04 |
| | | 20 | 4 | |
| | | 40 | 0 | 13.12 |
| | | 20 | 0 | |
| Glucose | M. antarcticus | 40 | 0 | 12.68 |
| Free Fatty | | 20 | 4 | |
| acids (FFA) | M. bullatus | 40 | 0 | 14.98 |
| | | 20 | 0 | |
| Glucose | M. antarcticus | 40 | 0 | 12.26 |
| Methyl | | 20 | 4 | |
| esters | M. bullatus | 40 | 0 | 15.73 |
| (FAME) | | 20 | 0 | |
| Glucose | M. antarcticus | 40 | 0 | 37.54 |
| Pomace oil | | 20 | 0, 2, 4, 6 | |
| | M. bullatus | 40 | 0 | 67.46 |
| | | 20 | 0, 2, 4, 6 | |

The production of MELs was assessed after the addition of the hydrophobic substrate. MELs began to precipitate in the form of firm and consistent MELs-rich beads, which were characterized based on their size and color. Again, for all the cases where vegetable oil, rich in integral triacylglycerols, was used as received, the formation of MELs-rich beads was a slow process. Moreover, those beads are never robustly formed as long there is a significant concentration of unhydrolyzed triacylglycerols in the fermentation broth.

Importantly, the assays reported on table 2 expand the use of other hydrophilic carbon sources beyond the use of D-glucose using M. bullatus or M. antarcticus strains and show that the addition of the hydrophobic source can be anticipated to the beginning of the fermentation with improvements in terms of speed for beads formation.

Moreover, the conditions considered confirm that, the use of lipidic derivatives of vegetable oil, i.e. (i) FFA enriched and (ii) FAME enriched substrates, promotes formation of MELs-rich beads in higher number, larger in size and sooner on the fermentation. The results obtained for maximum MELs concentrations as well as for the time at which MELs-rich beads are formed, and their characterization, are reported on Table 2 and FIG. 5. The results obtained show that the quality of MELs-rich beads is affected drastically by the choice of substrate and it does not relate solely to the concentration of MELs present in the fermentation broth. Importantly, one can observe that the period required for MELs-rich bead formation is shortened when the lipidic substrate is added initially (on day 0).

### Example 3

### MELs Production Using Multiple Steps for MELs-Rich Beads Recovery

### Culture Medium With Carbon Source:

Carbon source (variable)
KH₂PO₄, 0.3 g/L;
MgSO₄·7H₂O, 0.3 g/L;
Yeast extract, 1.0 g/L;

The culture medium for the fermentation process was prepared in sterile water, with initial pH equal to 6. The culture medium was inoculated with 10% (v/v) of preculture prepared in 1.A., and incubated under aerobic conditions and constant agitation at a temperature of 27° C. As for the carbon sources used, this strategy used a 40 g/L of D-glucose, a hydrophilic carbon source, present in the culture media, to support the first phase of cell growth and adaptation. Additionally, a hydrophobic carbon source was fed on day 4. This hydrophobic carbon source consisted of 20 g/L of a vegetable oil, rich on triacylglycerols, used as received or enzymatically pre-treated to decrease their content on triacylglycerols and increase its content on (i) FFA or (ii) FAME.

This process was assessed in shake flasks. Once the MELs-rich beads were form, they were collected and fresh substrate added. Such operation was made multiple times, repeatedly, as long as new beads were forming within a reasonable length of time. The beads compositions were analysed with GC (Gas Chromatography) and HPLC (High Performance Liquid Chromatography) methods.

The first set of MELs-rich beads was obtained only after 3 days of the initial lipid addition, either for FFA or for FAME enriched substrates. After the recovery of the MELs-rich bead, the culture cells proceeded to consume the newly introduced substrate and produced more MELs. However, with time, the period between the addition of the substrate and development of new MELs-rich beads increased. For FFA enriched substrates, time period increased from the initial 3 days to 6 days, and a total of 4 sets of MELs-rich beads were harvested. On the other hand, cultures fed with the FAME enriched substrate, produced beads for 5 times (one replica produced only 4), and the time periods between MELs-rich bead harvesting (from the addition of the lipid feed) were 3, 5, 5, 5, and 7 days.

Product to substrate yield (i.e. unit of product obtained per unit of substrate added) is an important parameter to assess process efficiency. Such yields were calculated to compare the efficiency of fermentations where a single MELs-rich beads harvest took place to the ones with multiple beads harvests. The total MELs obtained on the beads, used for yields estimation, was calculated considering the total dry mass of beads harvest and the respective MELs content. Data comparison of mass of beads collected, yields per unit of lipid substrate added, as well as total yields, is presented in Table 3.

TABLE 3
Fermentation parameters and MELs production performance
outputs for single and multiple bead recovery approaches
using FFA and FAME enriched substrates.

| Substrate | FFA | | FAME | |
|---|---|---|---|---|
| Fermentation duration | 12 | 24 | 12 | 29 |
| (days) | | | | |
| Total glucose added | 40 | 40 | 40 | 40 |
| (g/L) | | | | |
| Total lipids added (g/L) | 20 | 80 | 20 | 100 |
| Number of bead harvests | 1 | 4 | 1 | 5* |
| Total mass of MELs in | 4.490 | 20.857 | 6.530 | 27.572 |
| beads recovered (g/L) | | | | |
| MELs productivity (g of | 0.374 | 0.869 | 0.544 | 0.951 |
| MELs in beads/(L · day)) | | | | |
| Yield (mass of MELs in | 0.224 | 0.261 | 0.326 | 0.276 |
| beads/mass of lipids | | | | |
| added) | | | | |
| Total yield (mass of | 0.075 | 0.174 | 0.109 | 0.197 |
| MELs in beads/mass of | | | | |
| substrate added) | | | | |

Note:
When values are expressed by volume, that volume corresponds to the fermentation broth volume

### Example 4

### MELs-Rich Beads Production Using Olive Oil Pomace

### Culture Medium With Carbon Source:

Carbon source (variable)
KH₂PO₄, 0.3 g/L;
MgSO₄·7H₂O, 0.3 g/L;
Yeast extract, 1.0 g/L;

The culture medium for the fermentation process was prepared in sterile water, with initial pH equal to 6. The culture medium was inoculated with 10% (v/v) of M. antarcticus or M. bullatus pre-culture, prepared as described in 1.A. and incubated in bioreactor at a temperature of 27° C., and aerobic conditions controlled by an agitation cascade system Rushton, set up to maintain dissolved oxygen (DO) at a level of 20%, by varying agitation between 150-700 rpm, with a constant filtered air flux of 1 air volume per fermentation broth volume per minute (VVM).

As previously stated, lipid substrates enriched in free fatty acids and methyl esters increase the speed of substrate consumption by the yeast, as well as the formation of MELs-rich beads. In this regard, and in order to increase the sustainability of the described process, it was decided to assess a food industrial residue, olive pomace oil, which is constituted by 35.75% of monoacylglycerols, 45.28% of free fatty acids, 2.97% of diacylglycerols and 16% of triacylglycerols. This monoacylglycerol-enriched substrate, respects the previous threshold to contain less than 60% in triacylglycerols.

These fermentations started using a mixture of 40 g/L of D-glucose and 20 g/L of olive pomace oil, with further additions of 20 g/L of olive pomace oil at days 2, 4 and 6 of fermentation. MELs-rich beads formation started at day 4 of the fermentation and continued until day 8, showing that olive pomace oil is a good substrate for the production of MELs-enriched beads, attaining high yields at the end of the process (>60 g/L) as resumed in Table 4. In this case, the beads were olive green potentially due to specific molecules that are carried out from the olive pomace oil to the final product. This green color is distinct from the one previously observed in MELs-rich beads comprising high contents of residual lipids observed when triacylglycerol rich vegetable oils, such as soybean oil, were used as hydrophobic substrate.

TABLE 4
Fermentation parameters and MELs production performance
outputs fermentation using olive pomace oil.

Pomace oil
Day 9 of fermentation
Strains

| Parameters | M. bullatus | M. antarcticus |
|---|---|---|
| Biomass (g/L) | 45.00 | 43.00 |
| MELs (g/L) | 37.54 | 67.46 |
| Purity (%) | 55.38 | 89.71 |
| Yield(g/g) | 0.38 | 0.67 |
| Productivity | 4.17 | 6.75 |
| (g/(L · day)) | | |

### Example 5

### Design and Prototyping of the Tailored Non-Invasive Product Separation Device

The device features include (i) a grid with channels of width between 0.3 mm and 10 mm to retain large MELs-rich beads and allow fast fermentation broth, at high recirculation rates values, with minimal shear stress to avoid both cell stress and disruption as well as to mitigate smaller beads fragmentation; (ii) a chamber on the top of the grid of a size that allows collection of the total volume of beads formed in each collection cycle, that can have a transparent cap; (iii) one or multiple inlets to even supply of the aforementioned chamber, localized on the top of the grid, with the fermentation broth with MELs-rich beads; (iv) a chamber on the bottom side of the grid, opposite to the first chamber, to collect the filtrated culture media with viable cells, substrates and immature smaller MELs-rich beads, and; (v) one or several outlet in this bottom chamber to connect the device back to the bioreactor. Inlets and outlets will be also used to retrieve MELs-rich beads.

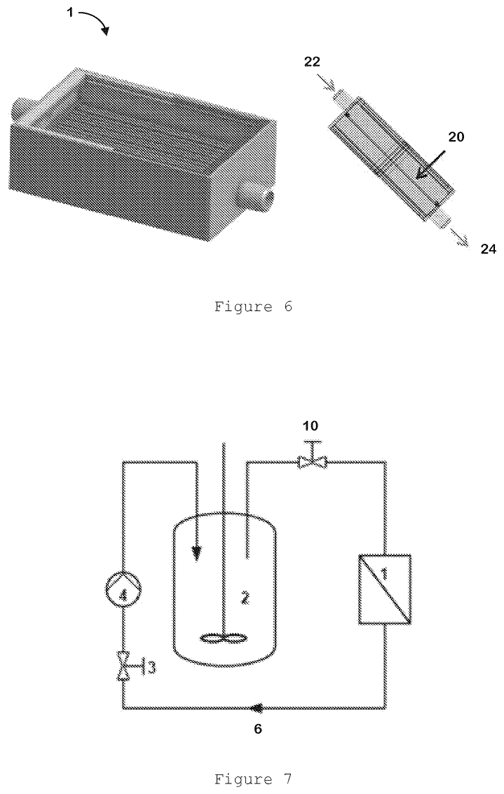

FIG. 6 represents a possible design of the non-invasive product separation device, which can be parallelepipedal or of other geometries.

The specific geometric parameters of the non-invasive product separation device can vary greatly without affecting its applicability. The dimensions of the device can be easily adapted to scale up the device to the desired size considering fermentation broth volume and amount of MELs produced. The geometry of the device can be also modified to facilitate adaptation the bioreactor, consider additional hydrodynamic features and grid support. The dimensions of the grids and the distance between them can be adapted to secure the recovery of beads of sufficient size (and thus of sufficient quality), and to secure undisturbed flow of the broth through the device. The device and the grid can be composed of a variation of materials-metals, plastics, etc., as long as they remain resistant to sterilization by different techniques, as well as show resistance to solvents which are eventually used for bead retrieval. Finally, due to the simplicity of the device, a design of a disposable device can be envisioned, which is discarded upon use and does not require sterilization as reuse is avoided.

### Example 6

### MELs Production Using the Tailored Non-Invasive Product Separation Devide to Recover MELs-Rich Beads and Recycle Culture Fermentation Broth

In the example of a fermentations using an initial addition of hydrophilic substrate, such as D-glucose, followed up by the addition of a hydrophobic substrate, such as waste cooking fried oil (WFO), a substrate rich in triacylglycerols, the beads usually appear at day 7-9 and disappear after day 10-11 of the fermentation. Note that those conditions do not correspond to the ones that provide higher MELs productivity nor higher MELs-rich beads quality, still they were selected to show the robustness of the approach to recover MELs-rich beads and further purification steps on more challenged fermentations as well as the relevance of the use of the non-invasive product separation device on broader scope.

In conventional approaches, the fermentation is not usually stopped when MELs-rich beads are formed, as the levels of unmetabolized lipids is still high. A conventional approach to address such issue, it is to extending the fermentation for a few more days increases the purity of the final product and higher titers can be obtained, typically implying longer equipment occupancy and sacrifice of productivity. The use of the non-invasive product separation device allows an alternative approach to address this challenge, as beads are recovery from the fermentation broth without interrupting the operation.

### Culture Medium:

Carbon source (variable)
KH₂PO₄, 0.3 g/L;
MgSO₄·7H₂O, 0.3 g/L;
Yeast extract, 1 g/L;

The culture medium for the fermentation process was prepared in sterile water, with initial pH equal to 6. The culture medium was inoculated with 10% (v/v) of M. antarcticus or M. bullatus preculture prepared in 1.A., corresponding to approx. 0.5 g/L of cell dry weight (CDW), incubated in a bioreactor at a temperature of 27° C. and aerobic conditions controlled by a Rushton turbine agitation cascade system set up to maintain a DO at a level of 20%, by varying agitation between 150-700 rpm with a constant filtered air flux of 1 VVM.

As for the carbon sources used, the substrate addition strategy included an initial media containing a mixture of 40 g/L of D-glucose and 20 g/L of WFO. Additionally, 20 g/L of WFO were fed on day 3 and another 20 g/L of WFO were fed at day 7, after recovery of the first lot of MELs-rich beads. The beads appear on day 5, increase their size on the following days. Two lots of MELs-rich beads were recovered, the first at day 7 and a second one at day 11.

The full integration process including cell circulation, MELs-rich beads recovery and retrieval are shown in FIG. 1. The stream circuit for the viable cell circuit between device (1) and bioreactor (2) is also shown in FIG. 7. The fermentation broth was pumped through external tubing system (first pathway 6) using a peristaltic pump (4) placed between the outlet of the device (1) and the bioreactor (2), in order to prevent damaging the beads caused by the pump. Using the viable cell circuit alone, the MELs-rich beads would need to be continuously accumulated in the device until the end of the fermentation, or removal of the device is required for MELs retrieval. Therefore, a second external tubing system (second pathway 7) was added to the system, as represented in FIG. 1, to connect the device (1) to a tank with methanol or warm water (reservoir 5) and such solution was pumped using a peristaltic pump (4) to retrieve MELs from the device.

The device prototype used in this case had 250 cm³ of internal volume (dimensions of 4×7×9 cm), with tubes leading in and out of the device with internal diameter of 1 cm. The metallic grid separated the device in two sections, with the section intended for bead collection being roughly 125 cm³. The flow rate was set at 0.5 L/min.

The first lot of MELs-rich beads recovered at day 7 yielded about 12 g/L (crude) of MELs with purity of 658. This MELs was produced from 40 g/L WFO (the 20 g/L of WFO initially added plus 20 g/L added at day 3). The second lot of MELs-rich beads was recovered on day 11 and yielded 8 g/L of MELs (crude) with 70% purity. The MELs on this second lot was produced from the 20 g/L of WFO added at days 7. Both lots of beads had similar water content at values of 33.4% and 26.338, for the first and second lots, respectively. Overall, in this specific example that comprises a fermentation of 11 days with two bead collections, the total MELs (crude) obtained in the beads was about 20 g/L with 67% of pure MELs in the form of granules, using 40 g/L of D-glucose and 60 g/L of WFO. The resulting yield is 0.134 g_{bead}MELs/g_{substrate}.

### Example 7

### Methods for MELs-Rich Bead Retrieval From Non-Invasive Product Separation Device

After the fermentation broth has been recirculated through the chamber of the product separation device for sufficient time, securing that the majority of the beads have bead recovered within the separation device, there are several methods for retrieval of the beads.

First, the beads can be physically removed from the device, since the device may be constructed with a slight modification in such a way to have one cover side that may be open and allow the removal of the beads. This is facilitated by the fact that the beads accumulated on the grid tend to merge into a larger solid structure. After removing the beads from the device, it can be sterilized and reattached to the closed system. This method results in the retrieval of the beads in their pure form, without water or solvents added.

Otherwise, an alternative method, which simplifies the process and reduces chances of contamination, is to include a separate pipeline for solvent circulation through the device, as presented in FIG. 1 (pathway 7). Warm water or organic solvents can be used to enable the MELs-rich beads complete retrieval from the device (1) to the reservoir (5). The amount of methanol needed for retrieving the MELs-rich beads from the device is relatively small (2:1) (v:w), and as an example only 100 mL of methanol was used to remove roughly 50 g of beads, obtained from 1 L fermentation broth, that is a volume ratio of 1:10 of methanol: fermentation broth. When warm water is used (>50° C.), the ratio of warm water to MELs-rich beads is around 10:1 warm water to fermentation broth ratio is around 1:5, thus corresponding to larger volumes of solubilizing agent in order to remove the beads from the device.

The method for retrieve the MELs-rich beads from the device upon their collection depends on the desired application of the MELs collected in this manner, namely, depending on whether the MELs are required to be solvent free or not, if they will be used in solid form, or are intended to be used in an aqueous solution.

### Example 8

### Separation of Triacylglycerols Using Methanol Precipitation

A mixture, comprising 38% MELs, 38% vegetable oil and 24% of smaller lipid derivatives typically found on crude MELs, was dissolved in methanol (6 g of mixture in 20 mL of methanol). The solution was vigorously mixed, and left to sediment for 30 minutes, after which a sedimented layer rich on the vegetable oil was formed, while the MELs and smaller lipidic derivatives remains dissolved on the methanol layer. The sedimented layer, rich on triacylglycerols was removed.

The amount of MELs, triacylglycerols and lipidic derivatives in the methanol phase were determined by GC.

Multiple steps of this method can be performed by adding fresh methanol to the sediment layer to reclaim the MELs co-precipitated with the triacyclglycerols. When the procedure was applied in three consecutive steps, MELs losses were 10% after the first step but reduced to 3.5% on cumulative losses after three steps. Overall, this method allows to remove 89% of the triacylglycerols rich vegetable oil with a single step.

The development of this efficient and simple method of separating triacylglycerols from the final product does not only increase the value of the product, but also increases the production efficiency, as larger feeds of vegetable oil can be used without raising concerns of residual unmetabolized triacylglycerols at the end of the fermentation because they can be removed and feed to the next fermentation cycle.

### Example 9

### Separation of Smaller Lipidic Contaminants From Methanol Solution Using Nanofiltration

Triacylglycerol presence can have a drastic impact on the final product quality, still the fermentation can be optimized to avoid residual triacylglycerols and to facilitate their complete hydrolysis. In such cases, the typically remaining lipidic contaminants are free fatty acids and which monoacylglycerols, are seldom used completely and often remain in the final product in varying concentrations. Alternatively, the use of methanol precipitation, as disclosed on the previous example, can be used to remove triacylglycerol contaminants from a MELs crude product. Also, for this case the MELs obtained at the methanol phase can be contaminated with free fatty acids and monoacylglycerols.

For the removal of these possible residual contaminants, one can explore the difference in the molecular weights of the molecules (MELs˜588-734 g/mol; Oleic acid˜282 g/mol; Glyceryl monooleate˜356 g/mol). For this case, organic solvent nanofiltration (OSN) can be applied using an OSN membrane with adequate molecular weight cut-off (MWCO); where MWCO is defined as molecular size of the solute which membrane rejection is 90% (Wu et al. 2017). Yet, the molecular weight range of 356 to 588 g/mol is narrow, and attempts to use commercially available membranes to perform this separation have failed.

The OSN membranes used were made in-house by phase inversion from a casting solution of polybenzimidazole (PBI) dissolved in in dimethylacetamide (DMAc). The PBI solutions were casted using a casting knife with a 250-micrometer gap and phase inverted in water, then transfer to an isopropanol solution and finally preconditioned with methanol before use. 22%, 24% and 26% PBI solutions in DMAc were casted in films and phase inverted as described above o obtain different membranes of different profiles of solute rejections and assessed for separation of MELs and lipids. The obtained membranes were labelled as 22% PBI, 24% PBI and 26% PBI according with the PBI content on casting solution.

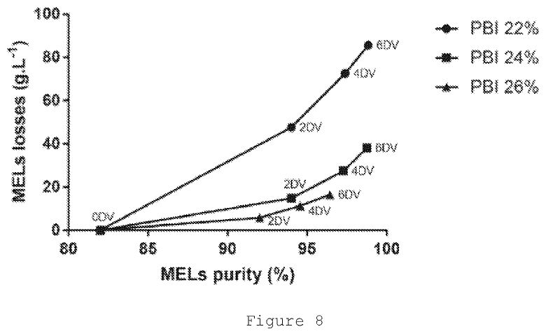

Solutions in methanol of 50 g/L crude MELs, containing small lipidic impurities (FFA and monoacylglicerols), were submitted to diafiltrations in a dead-end filtration setup pressurized at 15 bar, where methanol is added to the pressurized system and the filtrate is collected in order to maintain a constant volume in the filtration cell. The addition of fresh methanol on a volume that equals the initial solution submitted to a diafiltration corresponds to one diavolume (DV). The MELs purity and MELs losses using the three aforementioned PBI membranes were estimated for 2, 4 and 6 diavolumes (DV). These results are presented in FIG. 8.

The use of the 22% PBI membrane resulted in significant losses of MELs. Those losses were not expected as initial experimental characterization of the 22% PBI membrane estimate membrane rejections for a 580 g/mol polystyrene and a 1017 g/mol Rose Bengal dye to be 91%and 100%, respectively, and the molecular weights of MELs are in this range. The use of 26% PBI membrane obtained a satisfactory purity (˜92%) with minimal losses (˜5%) using just two diavolumes. For the same conditions, the 24% PBI membrane generated MELs with slightly better purity (94%), however with three times higher MELs losses (15%).

The high removal of FFA and monoacylglycerol obtained using the 26% PBI membrane was surprising, as previous studies show a membrane made from 26 wt. % PBI solution to present high rejections of 95% for Chlorhexidine (505 g/mol), but also relatively high rejections at values of 50-65% for 4-chloroaniline (127 g/mol), a compound much smaller than the FFA or monoacylglycerol. These results are non-trivial, in particular considering the polydispersity of MELs size, existence of molecular isoforms, potential intermolecular interactions, and typical pore size dispersity in nanofiltration membranes.

However, considering the goal to provide sustainable solvent-efficient downstream processing system, the performance of the 26% PBI membrane efficiency using only 2DV is quite significant and unique, in particular considering the separation of molecules with narrow molecular weight differences, calculated at values of 182 g/mol to 394 g/mol considering, respectively, the molecular weight difference between glyceryl monooleate and the MELs isoform of smaller molecular weight or between oleic acid and the MELs isoform of higher molecular weight.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. Device (1) for macrofiltration comprising a grid (20), the grid comprising a plurality of channels, each channel having a width between 0.3 mm and 10 mm; the grid separating a first chamber and a second chamber each chamber positioned on an opposite side of said grid (20); at least one inlet (22) and at least one outlet (24), wherein:
said at least one inlet (22) is configured to evenly supply a fluid to said first chamber;
said second chamber is configured to collect a filtrated fluid from the first chamber; and
said at least one outlet (24) is configured to connect the second chamber to a bioreactor.

2. Device according to claim 1, wherein said macrofiltration comprises mannosylerythritol lipids (MELs) recovery.

3. Device according to claim 2, wherein said MELs are in the form of particles, said particles being retained in said first chamber.

4. System comprising the device according to claim 1.

5. System according to claim 4, further comprising a bioreactor (2).

6. System according to claim 5, comprising a first pathway (6) in fluid communication, wherein said first pathway comprises sequentially a first valve (3), a first pump (4), said bioreactor (2) and a second valve (10), where the first pathway (6) is positioned between said at least one outlet (24) and inlet (22) of the device (1).

7. System according to claim 4, further comprising a reservoir (5).

8. System according to claim 7, comprising a second pathway (7) in fluid communication, wherein said second pathway comprises sequentially a third valve (12), a second pump (8), said reservoir (5) and a fourth valve (14), where the second pathway (7) is positioned between said at least one outlet (24) and inlet (22) of the device (1).

9. System according to claim 4, comprising: a bioreactor (2), a reservoir (5), a first pathway (6) and a second pathway (7), wherein:
said device (1) comprises at least one outlet (24) in fluid communication with said first pathway (6) and said second pathway (7), and at least one inlet (22) in fluid communication with said first pathway (6) and said second pathway (7);
said first pathway (6) comprises sequentially a first valve (3), a first pump (4), said bioreactor (2) and a second valve (10), positioned between said at least one outlet and inlet of the device; and
said second pathway (7) comprises sequentially a third valve (12), a second pump (8), said reservoir (5) and a fourth valve (14), positioned between said at least one outlet and inlet of the device.

10. Process for enhancing mannosylerythritol lipids (MELs) production, comprising the steps of:
a) contacting between 1% and 70% weight per weight (w/w) of a substrate and between 0.01% and 10% of a yeast in a culture medium solution under fermentation conditions thereby producing MELs in a fermentation broth;
b) separating said MELs from the fermentation broth, said solution optionally comprising between 4 and 150 (g/L) of said MELs; and
c) washing said MELs with a second solution, said second solution optionally comprising an alcoholic solution and/or an aqueous solution, wherein the ratio of said second solution, to said fermentation broth is between 3:10 to 1:50 volume per volume (v/v);
optionally further comprising a step of removal of tryacylglicerols by precipitation in methanol, while said MELs remain soluble;
optionally further comprising a step of nanofiltering said MELs solution, particularly wherein said MELs have a purity of more than 90%;
wherein said MELs optionally has a purity of more than 60%.

11. (canceled)

12. Process according to claim 10, performed in the device of claim 1 or in the system of any one of claims 2 to 9.

13. Process according to claim 10, further comprising a step of circulating said fermentation broth solution from step b), back to step a).

14. (canceled)

15. Process according to claim 10, wherein said MELs is in the form of particles, optionally wherein said particles have a particle size of more than 1 mm, particularly between 1 mm and 50 mm.

16. (canceled)

17. (canceled)

18. Process according to claim 10, wherein said substrate comprises a hydrophilic carbon source, a hydrophobic carbon source, or both, particularly wherein said hydrophilic carbon source comprises an alcohol, a sugar or any combination thereof; or said hydrophilic carbon source comprises a carbon rich agricultural, industrial waste or residue, selected from the group comprising: cheese whey, wheat straw, sugar beet residues, sugar cane residues, crude glycerol, or any combination thereof; or said hydrophobic carbon source comprises an oil rich in triacylglycerol, selected from the group comprising: soybean oil, rapeseed oil, waste cooking fried oil, animal fat, and algae driven oil; or wherein said hydrophobic carbon source comprises free fatty acids, monoacylglycerol, diacylglycerol, or any combination thereof, particularly wherein said hydrophobic carbon source is rich in free fatty acids and/or mono/di acylglycerol mixtures, obtained without dedicated pre-treatment or obtained from pre-treatment of triacylglycerols after their hydrolysis or transesterification.

19. (canceled)

20. (canceled)

21. (canceled)

22. (canceled)

23. (canceled)

24. Process according to claim 18, wherein said hydrophobic carbon source is added in step a), or up to 6 days after step a), particularly wherein said hydrophobic carbon source and said hydrophilic carbon source are present in a ratio ranging from 10:1 to 1:1 by weight, respectively.

25. (canceled)

26. (canceled)

27. (canceled)

28. (canceled)

29. (canceled)

30. Composition comprising a plurality of mannosylerythritol lipids (MELs), wherein said MELs is in the form of particles.

31. Use of the device of claim 1, for production and/or purification of mannosylerythritol lipids (MELs).

32. Use of the system of claim 4, for production and/or purification of mannosylerythritol lipids (MELs).

33. Use of the process of claim 10, for production and purification of mannosylerythritol lipids (MELs).

34. Use of the composition of claim 30, in agriculture, food, beverages, cleaning products, bioremediation, oil and gas industry, bio and fine chemical industry, cosmetics, pharmaceutical industry, or any combination thereof.
